(19) United States
(12) Patent Application Publication
(10) Pub. No.: US 2019/0261858 A1
(43) Pub. Date: Aug. 29, 2019

(54) IMAGING METHOD FOR ACUTE LYMPHOBLASTIC LEUKEMIA USING AN IMAGING AGENT AND METHOD OF PRODUCING AN IMAGING AGENT

(71) Applicants: Stanley Satz, Lake Worth, FL (US); Roseanne Satz, Lake Worth, FL (US)

(72) Inventors: Stanley Satz, Lake Worth, FL (US); Roseanne Satz, Lake Worth, FL (US)

(21) Appl. No.: 16/176,352

(22) Filed: Oct. 31, 2018

Related U.S. Application Data
(60) Provisional application No. 62/580,675, filed on Nov. 2, 2017.

(51) Int. Cl. A61B 5/00; A61K 49/22; A61B 5/055; A61B 90/00

(52) U.S. Cl. CPC A61B 5/0033; A61B 5/0071; C12Q 1/6886; A61B 5/055; A61B 90/39; A61K 49/222

(57) ABSTRACT

The present invention provides an in vivo imaging method that facilitates the diagnosis of Acute Lymphoblastic Leukemia (ALL) at an early stage. Early diagnosis is particularly advantageous as a tool to select more aggressive therapy, to estimate the success rate for visualizing ALL at the time of diagnosis.

### BACKGROUND OF THE INVENTION

### 1. Technical Field of the Invention

The present invention relates to in vivo imaging and in particular to an in vivo imaging method to facilitate the early diagnosis of Acute Lymphoblastic Leukemia (ALL) in pediatric patients.

### 2. Brief Description of the Related Art

Leukemia is a cancer that begins in the bone marrow (the soft inner part of some bones), but in most cases, moves into the blood. It can then spread to other parts of the body, such as organs and tissues. ALL, one of the four main types of leukemia, is a slow-growing blood cancer that starts in bone marrow cells called lymphocytes, or white blood cells. Once these white blood cells are affected by leukemia, they do not go through their normal process of maturing. The lymphocytes continue to reproduce and build up, invade the blood quickly. ALL is an aggressive type of leukemia; without treatment, most patients with acute leukemia would live only a few months (American Cancer Society 2014).

According to The Surveillance, Epidemiology and End Results program (SEER) and Orphanet, ALL is a malignant proliferation of lymphoid cells blocked at an early stage of differentiation and accounts for 75% of all cases of childhood leukemia. About 3,000 children in the United States and 5,000 children in Europe are diagnosed with ALL each year. The peak incidence occurs between 2 and 5 years of age.

ALL is biologically heterogeneous and morphologic, immunologic, cytogenetic, biochemical, and molecular genetic characterization of leukemia lymphoblasts is needed to establish the diagnosis or to exclude other possible causes of bone marrow failure and, finally, to classify ALL subtypes. Biological findings include hyperleukocytosis due to circulating lymphoblasts, anemia and thrombocytopenia. Diagnosis is established by bone marrow biopsy revealing leukemic cell infiltration. The chemotherapy protocols adopted by international cooperative groups have four main objectives: induction with the aim of complete remission, preventative therapy to avoid central nervous system involvement, consolidation/re-induction, and maintenance therapy. Although management of relapse remains largely controversial, high dose chemotherapy blocks and stem cell transplantation are approaches increasingly adopted in most cases (Orphanet 2012).

PET/CT imaging requires less time than 123I SPECT and exhibits two to three orders of magnitude higher sensitivity than SPECT. The higher spatial resolution of PET as compared to SPECT is 3-6 mm versus 10-15 mm, providing superior image quality, better visualization of small lesions. Moreover, [18F]-FLT has broad spectrum higher affinity for angiogenic tumors than [18F]-FDG.

Computerized tomography scan (also called a CT or CAT scan)—a diagnostic imaging procedure that uses a combination of x-rays and computer technology to produce cross-sectional images (often called slices), both horizontally and vertically, of the body. CT scans are more detailed than general x-rays.

Magnetic resonance imaging (MRI)—a diagnostic procedure that uses a combination of large magnets, radiofrequencies and a computer to produce detailed images of organs and structures within the body.

MRI provides greater anatomical detail than CT scan and does a better job of distinguishing between tumors, tumor-related swelling and normal tissue.

Magnetic resonance spectroscopy (MRI)—a diagnostic test conducted along with an MRI. It can detect the presence of organic compounds around the tumor tissue that can identify the tissue as normal or tumor, and may also be able to tell if the tumor is a glial tumor or if it is of neuronal origin (originating in a neuron, instead of an astrocytic or glial cell).

Although the above-described in vivo imaging techniques may overcome the problem of inaccurate differential diagnosis and inappropriate application of ALL treatment, they all target the disease process at a stage of cell proliferation in bone marrow.

### SUMMARY OF THE INVENTION

The present invention provides an in vivo imaging method that facilitates the diagnosis of Acute Lymphoblastic Leukemia (ALL) at an early stage. Early diagnosis is particularly advantageous as a tool to select more aggressive therapy, to estimate the success rate for visualizing ALL at the time of diagnosis. A further advantage of the present invention over the prior art is that the in vivo imaging agent. FLT uptake has been shown to correlate with pathology-based proliferation measurements, including the Ki-67 score, in a variety of human cancers.

### BRIEF DESCRIPTION OF THE DRAWING FIGURE

FIG. 1 is a flow diagram depicting a preferred embodiment of the imaging method according to the invention

### DETAILED DESCRIPTION OF THE INVENTION

### Method of Imaging

In one aspect, the present invention provides an in vivo imaging agent for use in a method to determine the presence of, or susceptibility to, Acute Lymphoblastic Leukemia, wherein said in vivo imaging agent comprises [18F]FLT, a lipophilic azomycin-based cell sensitizer labelled with an in vivo imaging moiety. The compound, 3′-deoxy-3′-fluorothymidine (FLT) is a nucleoside analog that enters cells and is phosphorylated by human thymidine kinase 1, but the 3′ substitution prevents further incorporation into DNA. According to a preferred implementation, the method may be carried out comprising the steps of:

(i) administering to a subject a detectable quantity of said in vivo imaging agent;

(ii) allowing said administered in vivo imaging agent of step (i) to bind covalently to cellular molecules at rates that are inversely proportional to and phosphorylated by human thymidine kinase 1 in the DNA;

(iii) detecting signals emitted by said bound in vivo imaging agent of step (ii) using an in vivo imaging method;

(iv) generating an image representative of the location and/or amount of said signals; and, (v) using the image generated in step (iv) to determine of the presence of, or susceptibility to, ALL.

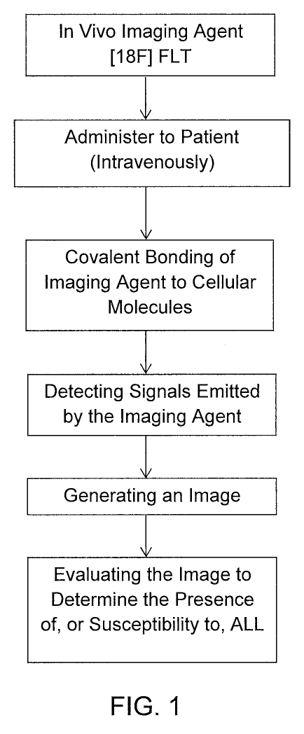

FIG. 1 depicts a flow diagram illustrative of an exemplary implementation of the method for determining the presence of, or susceptibility to, ALL. In the exemplary illustration a preferred implementation of the method according to the invention depicts an in vivo imaging agent that includes an in vivo imaging moiety comprising a 3′ substituted nucleoside analog, which is represented as an ¹⁸F nucleoside analog, and more particularly as 3′-deoxy-3′-¹⁸F-fluorothymidine (18F-FLT).

The in vivo imaging moiety is preferably chosen from: (i) a radioactive metal ion;

(ii) a paramagnetic metal ion; (iii) a gamma-emitting radioactive halogen; (iv) a positron-emitting radioactive non-metal; (v) a reporter suitable for in vivo optical imaging. In vivo imaging agents may be conveniently prepared by reaction of a precursor compound with a suitable source of the in vivo imaging moiety. A “precursor compound” comprises a derivative of the in vivo imaging agent, designed so that chemical reaction with a convenient chemical form of the in vivo imaging moiety occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. The precursor compound may optionally comprise a protecting group for certain functional groups of the precursor compound.

When the in vivo imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; or γ-emitters such as ^{99m}Tc, ¹¹¹In, ^{113m}In, or ⁶⁷Ga, and when the in vivo imaging moiety is a positron-emitting radioactive non-metal, a suitable positron-emitting radioactive non-metal may be ¹¹C, ¹³N, ¹⁵O, ¹⁷F, ¹⁸F, ⁷⁵Br, ⁷⁶Br or ¹²⁴I, with the preferred non-metal positron emitter being 18F.

When the imaging moiety comprises a metal ion, it is preferably present as a metal complex of the metal ion with a synthetic ligand. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include other excipients in the preparation in vitro (e.g. radioprotectants or antimicrobial preservatives used in the preparation), or endogenous compounds in vivo (e.g. glutathione, transferrin or plasma proteins). The term “synthetic” has its conventional meaning, i.e. man-made as opposed to being isolated from natural sources e.g. from the mammalian body. Such compounds have the advantage that their manufacture and impurity profile can be fully controlled.

The method of the invention begins by administering a detectable quantity of an in vivo imaging agent to a subject. Since the ultimate purpose of the method is the provision of a diagnostically-use ml image, administration to the subject of said in vivo imaging agent can be understood to be a preliminary step necessary for facilitating generation of said image. In an alternative embodiment the method of the invention can be said to begin by providing a subject to whom a detectable quantity of an in vivo imaging agent has been administered. “Administering” the in vivo imaging agent means introducing the in vivo imaging agent into the subject's body, and is preferably carried out parenterally, most preferably intravenously. The intravenous route represents the most efficient way to deliver the in vivo imaging agent throughout the body of the subject.

The “subject” of the invention is preferably a mammal, most preferably an intact mammalian body in vivo. In an especially preferred embodiment, the subject of the invention is a human.

The term “in vivo imaging agent” broadly refers to a compound which can be detected following its administration to the mammalian body in vivo. The in vivo imaging agent of the present invention comprises a lipophilic azomycin-based hypoxic cell sensitizer labelled with an in vivo imaging moiety. The term “labelled with an in vivo imaging moiety” means either (i) that a particular atom of the lipophilic azomycin-based hypoxic cell sensitizer is an isotopic version suitable for in vivo detection, or (ii) that a group comprising said in vivo imaging moiety is conjugated to said lipophilic azomycin-based hypoxic cell sensitizer.

The “detection” step of the method of the invention involves the detection of signals either externally to the human body or via use of detectors designed for use in vivo, such as intravascular radiation or optical detectors such as endoscopes (e.g. suitable for detection of signals in the gut), or radiation detectors designed for intra-operative use. This detection step can also be understood as the acquisition of signal data. The “in vivo imaging method” selected for detection of signals emitted by said in vivo imaging moiety depends on the nature of the signals. Therefore, where the signals come from a paramagnetic metal ion, magnetic resonance imaging (MRI) is used, where the signals are gamma rays, single photon emission tomography (SPECT) is used, where the signals are positrons, positron emission tomography (PET) is used, and where the signals are optically active, optical imaging is used. All are suitable for use in the method of the present invention, with PET and SPECT are preferred, as they are least likely to suffer from background and therefore are the most diagnostically useful.

The in vivo imaging agent of the invention is preferably administered as a “radiopharmaceutical composition” which comprises said in vivo imaging agent, together with a biocompatible carrier, in a form suitable for mammalian administration.

The “biocompatible carrier” is a fluid, especially a liquid, in which the in vivo imaging agent as defined herein is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilize more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm volume) which contains multiple patient doses, whereby single patient doses can be withdrawn into clinical grade syringes at various time intervals during the viable lifetime of the preparation to suit the clinical situation. Pre-filled syringes are designed to contain a single human dose, or “unit dose”, and are therefore preferably a disposable or other syringe suitable for clinical use.

Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceutical composition may be prepared from a kit. Alternatively, it may be prepared under aseptic manufacture conditions to give the desired sterile product. The pharmaceutical composition may also be prepared under non-sterile conditions, followed by terminal sterilization using e.g. gamma-irradiation, autoclaving, dry heat or chemical treatment (e.g. with ethylene oxide).

### Selected Methods of the Invention

One aspect of the invention relates to a method for FLT imaging a subject suffering from Acute Lymphoblastic Leukemia condition, comprising the step of:

administering a compound, or a composition comprising a pharmaceutically acceptable carrier and the compound or a pharmaceutically acceptable salt thereof, to the subject, wherein the compound is represented by formula I-IV:

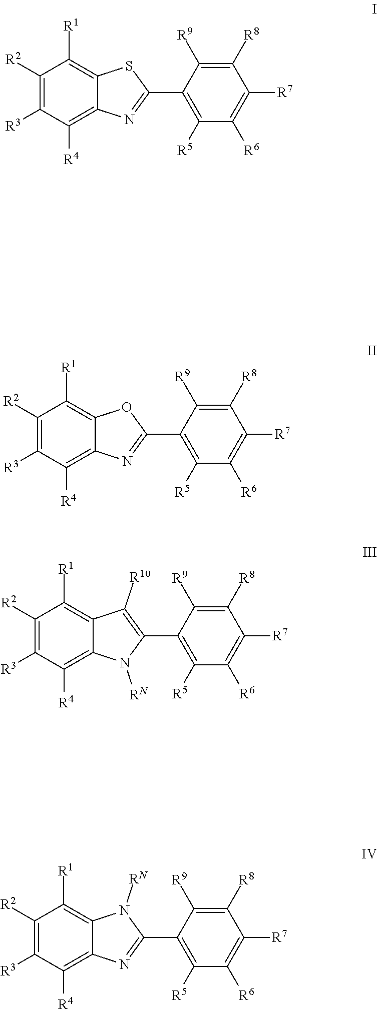

wherein, independently for each occurrence,

R.sup.1, R.sup.2, R.sup.3, R.sup.4, R.sup.5, R.sup.6, R.sup.7, R.sup.8, R.sup.9, and R.sup.10 are hydrogen, halo, azido, alkyl, haloalkyl, perhaloalkyl, fluoroalkyl, perfluoroalkyl, aralkyl, alkenyl, alkynyl, cycloalkyl, heterocyclyl, aryl, heteroaryl, aralkyl, heteroaralkyl, hydroxy, alkoxy, aryloxy, heteroaryloxy, aralkyloxy, heteroaralkyloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfo, sulfonate, sulfonyl, sulfonamido, formyl, cyano, isocyano, or —Y-(haloalkylene)-alkyl;

R.sup.N is hydrogen, lower alkyl, or -(haloalkylene)-alkyl;

Y is a bond, N(R.sup.N), O, or S;

provided that at least one of R.sup.1, R.sup.2, R.sup.3, R.sup.4, R.sup.5, R.sup.6, R.sup.7, R.sup.8, R.sup.9, or R.sup.10 is —Y-(haloalkylene)-alkyl; or R.sup.N is -(haloalkylene)-alkyl.

In certain embodiments, the present invention relates to the aforementioned method and any of the attendant definitions, wherein the compound is represented by formula I.

In certain embodiments, the present invention relates to the aforementioned method and any of the attendant definitions, wherein the compound is represented by formula II.

In certain embodiments, the present invention relates to the aforementioned method and any of the attendant definitions, wherein the compound is represented by formula III.

In certain embodiments, the present invention relates to the aforementioned method and any of the attendant definitions, wherein the compound is represented by formula IV.

In certain embodiments, the present invention relates to the aforementioned method and any of the attendant definitions, wherein R.sup.7 is —Y-(haloalkylene)-alkyl.

In certain embodiments, the present invention relates to the aforementioned method and any of the attendant definitions, wherein R.sup.7 is —Y-(fluoroalkylene)-alkyl.

In certain embodiments, the present invention relates to the aforementioned method and any of the attendant definitions, wherein R.sup.7 is —Y-(monofluoroalkylene)-alkyl.

In certain embodiments, the present invention relates to the aforementioned method and any of the attendant definitions, wherein R.sup.7 is —Y-(monofluoroalkylene)-alkyl; and said fluoro substituent is bound to a secondary alkylene carbon.

In certain embodiments, the present invention relates to the aforementioned method and any of the attendant definitions, wherein R.sup.7 is —Y-(monofluoroalkylene)-alkyl; and said fluoro substituent is bound to a tertiary alkylene carbon.

In certain embodiments, the present invention relates to the aforementioned method and any of the attendant definitions, wherein R.sup.7 is —Y—([F-18] fluoroalkylene)-alkyl.

In certain embodiments, the present invention relates to the aforementioned method and any of the attendant definitions, wherein R.sup.7 is —Y—([F-18] monofluoroalkylene)-alkyl; and said [F-18] fluoro substituent is bound to a secondary alkylene carbon.

In certain embodiments, the present invention relates to the aforementioned method and any of the attendant definitions, wherein R.sup.7 is —Y—([F-18] monofluoroalkylene)-alkyl; and said [F-18] fluoro substituent is bound to a tertiary alkylene carbon.

In certain embodiments, the present invention relates to the aforementioned method and any of the attendant definitions, wherein R.sup.7 is —Y—(CH.sub.2CH.sup.18F)—CH.sub.3.

In certain embodiments, the present invention relates to the aforementioned method and any of the attendant definitions, wherein Y is N(R.sup.N).

In certain embodiments, the present invention relates to the aforementioned method and any of the attendant definitions, wherein R.sup.N is hydrogen.

In certain embodiments, the present invention relates to the aforementioned method and any of the attendant definitions, wherein R.sup.1, R.sup.3, R.sup.4, R.sup.5, R.sup.6, R.sup.8, and R.sup.9 are hydrogen.

In certain embodiments, the present invention relates to the aforementioned method and any of the attendant definitions, wherein R.sup.2 is hydroxy, alkoxy, aryloxy, heteroaryloxy, aralkyloxy, or heteroaralkyloxy.

In certain embodiments, the present invention relates to the aforementioned method and any of the attendant definitions, wherein R.sup.2 is hydroxy.

In certain embodiments, the present invention relates to the aforementioned method and any of the attendant definitions, wherein R.sup.10 is hydrogen.

In certain embodiments, the present invention relates to the aforementioned method and any of the attendant definitions, wherein the compound is represented by formula I; R.sup.7 is —N(H)-(monofluoroalkylene)-CH.sub.3; R.sup.1, R.sup.3, R.sup.4, R.sup.5, R.sup.6, R.sup.8 and R.sup.9 are hydrogen; and R.sup.2 is hydroxy.

In certain embodiments, the present invention relates to the aforementioned method and any of the attendant definitions, wherein the compound is represented by formula I; R.sup.7 is —N(H)—([F-18] monofluoroalkylene)-CH.sub.3; R.sup.1, R.sup.3, R.sup.4, R.sup.5, R.sup.6, R.sup.8 and R.sup.9 are hydrogen; and R.sup.2 is hydroxy.

In certain embodiments, the present invention relates to the aforementioned method and any of the attendant definitions, wherein the compound is represented by formula I; R.sup.7 is —N(H)—(CH.sub.2 CHF)—CH.sub.3; R.sup.1, R.sup.3, R.sup.4, R.sup.5, R.sup.6, R.sup.7, and R.sup.8 are hydrogen; and R.sup.2 is hydroxy.

In certain embodiments, the present invention relates to the aforementioned method and any of the attendant definitions, wherein the compound is represented by formula I; R.sup.7 is —N(H)—(CH.sub.2CH.sup.18F)—CH.sub.3; R.sup.1, R.sup.3, R.sup.4, R.sup.5, R.sup.6, R.sup.8, and R.sup.9 are hydrogen; and R.sup.2 is hydroxy.

In certain embodiments, the present invention relates to the aforementioned method and any of the attendant definitions, wherein the compound is

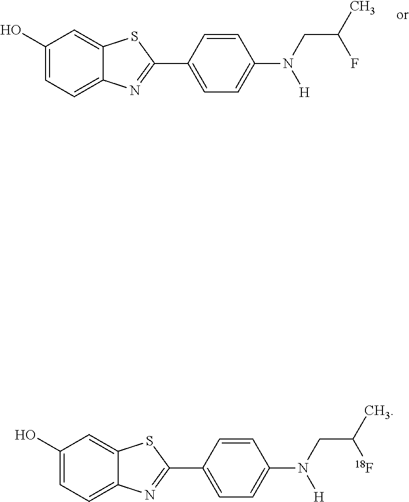 [SMILES: CC(F)CNc1ccc(-c2nc3ccc(O)cc3s2)cc1 ; CC([18F])CNc1ccc(-c2nc3ccc(O)cc3s2)cc1]

In certain embodiments, the present invention relates to the aforementioned method and any of the attendant definitions, wherein the pharmaceutical composition further comprises a sugar alcohol.

In certain embodiments, the present invention relates to the aforementioned method and any of the attendant definitions, wherein the pharmaceutical composition further comprises glycol, glycerol, erythritol, arabitol, xylitol, ribitol, mannitol, sorbitol, isomalt, maltitol, or lactitol.

In certain embodiments, the present invention relates to the aforementioned method and any of the attendant definitions, wherein the pharmaceutical composition further comprises mannitol.

Another aspect of the invention relates to a method for 18F-FLT imaging a subject suffering from Acute Lymphoblastic Leukemia condition, comprising the step of:

administering a compound, or a composition comprising a pharmaceutically acceptable carrier and the compound or a pharmaceutically acceptable salt thereof, to the subject, wherein the compound is represented by formula I-IV:

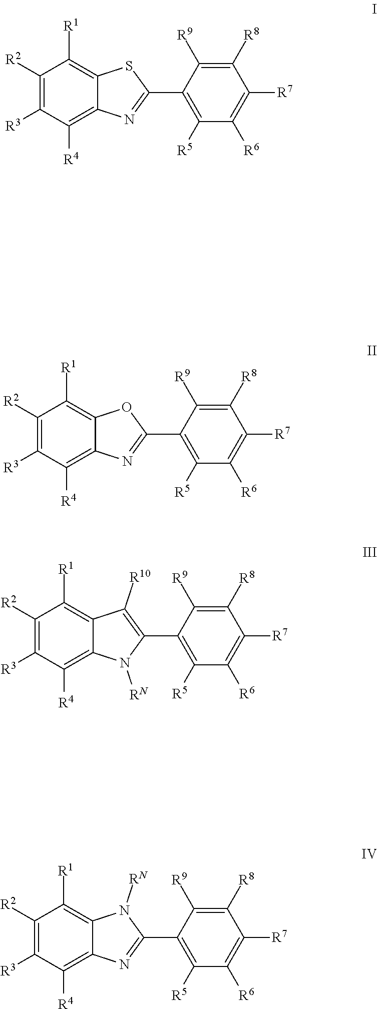

wherein, independently for each occurrence,

R.sup.1, R.sup.2, R.sup.3, R.sup.4, R.sup.5, R.sup.6, R.sup.8, R.sup.9, and R.sup.10 are hydrogen, halo, azido, alkyl, haloalkyl, perhaloalkyl, fluoroalkyl, perfluoroalkyl, aralkyl, alkenyl, alkynyl, cycloalkyl, heterocyclyl, aryl, heteroaryl, aralkyl, heteroaralkyl, hydroxy, alkoxy, aryloxy, heteroaryloxy, aralkyloxy, heteroaralkyloxy, amino, alkylamino, arylamino, acylamino, heteroarylamino, nitro, sulfhydryl, imino, amido, phosphonate, phosphinate, acyl, carboxyl, oxycarbonyl, aryloxy, silyl, thioether, sulfo, sulfonate, sulfonyl, sulfonamido, formyl, cyano, or isocyano;

R.sup.7 is —Y-(haloalkylene)-R;

Y is a bond, N(R.sup.N), O, or S;

R.sup.N is hydrogen, or lower alkyl; and R is

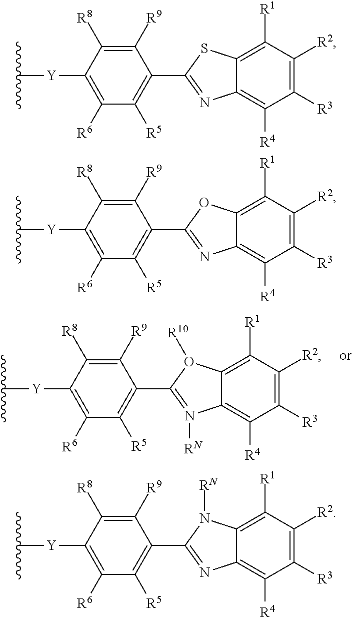

In certain embodiments, the present invention relates to the aforementioned method and any of the attendant definitions, wherein the compound is represented by formula I.

In certain embodiments, the present invention relates to the aforementioned method and any of the attendant definitions, wherein the compound is represented by formula II.

In certain embodiments, the present invention relates to the aforementioned method and any of the attendant definitions, wherein the compound is represented by formula III.

In certain embodiments, the present invention relates to the aforementioned method and any of the attendant definitions, wherein the compound is represented by formula IV.

In certain embodiments, the present invention relates to the aforementioned method and any of the attendant definitions, wherein R.sup.7 is —Y-(haloalkylene)-Y—R.

In certain embodiments, the present invention relates to the aforementioned method and any of the attendant definitions, wherein R.sup.7 is —Y-(fluoroalkylene)-Y—R.

In certain embodiments, the present invention relates to the aforementioned method and any of the attendant definitions, wherein R.sup.7 is —Y-(monofluoroalkylene)-Y—R.

In certain embodiments, the present invention relates to the aforementioned method and any of the attendant definitions, wherein R.sup.7 is —Y—([F-18] fluoroalkylene)-Y—R.

In certain embodiments, the present invention relates to the aforementioned method and any of the attendant definitions, wherein R.sup.7 is —Y—([F-18] monofluoroalkylene)-Y—R.

In certain embodiments, the present invention relates to the aforementioned method and any of the attendant definitions, wherein Y is N(R.sup.N).

In certain embodiments, the present invention relates to the aforementioned method and any of the attendant definitions, wherein R.sup.N is hydrogen.

In certain embodiments, the present invention relates to the aforementioned method and any of the attendant definitions, wherein R.sup.7 is —N(H)-(haloalkylene)-N(H)—R.

In certain embodiments, the present invention relates to the aforementioned method and any of the attendant definitions, wherein R.sup.1, R.sup.3, R.sup.4, R.sup.5, R.sup.6, R.sup.6, R.sup.8, and R.sup.9 are hydrogen.

In certain embodiments, the present invention relates to the aforementioned method and any of the attendant definitions, wherein R.sup.2 is hydroxy, alkoxy, aryloxy, heteroaryloxy, aralkyloxy, or heteroaralkyloxy.

In certain embodiments, the present invention relates to the aforementioned method and any of the attendant definitions, wherein R.sup.2 is hydroxy.

In certain embodiments, the present invention relates to the aforementioned method and any of the attendant definitions, wherein R.sup.10 is hydrogen.

In certain embodiments, the present invention relates to the aforementioned method and any of the attendant definitions, wherein the compound is represented by formula I; R.sup.7 is —Y-(haloalkylene)-Y—R; and R is

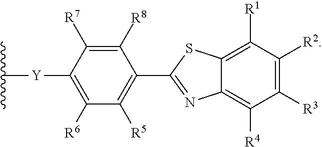

In certain embodiments, the present invention relates to the aforementioned method and any of the attendant definitions, wherein the compound is represented by formula II; wherein R.sup.7 is —Y-(haloalkylene)-Y—R; and R is

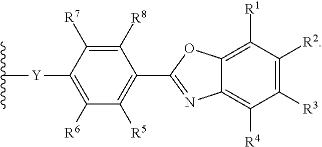

In certain embodiments, the present invention relates to the aforementioned method and any of the attendant definitions, wherein the compound is represented by formula III; R.sup.7 is —Y-(haloalkylene)-Y—R; and R is

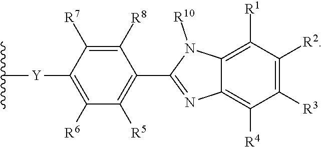

In certain embodiments, the present invention relates to the aforementioned method and any of the attendant definitions, wherein the compound is represented by formula IV; wherein R.sup.7 is —Y-(haloalkylene)-Y—R; and R is

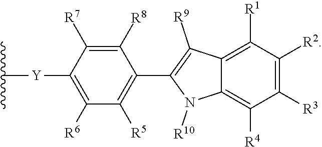

In certain embodiments, the present invention relates to the aforementioned method and any of the attendant definitions, wherein the compound is represented by formula I; wherein R.sup.7 is —N(H)-(monofluoroalkylene)-N(H)—R; R is

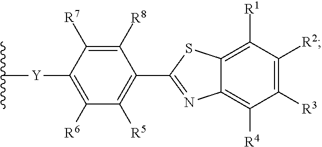

R.sup.1, R.sup.3, R.sup.4, R.sup.5, R.sup.6, R.sup.8, and R.sup.9 are hydrogen; and R.sup.2 is hydroxy.

In certain embodiments, the present invention relates to the aforementioned method and any of the attendant definitions, wherein the compound is represented by formula I; R.sup.7 is —N(H)—([F-18] monofluoroalkylene)-N(H)—R; R is

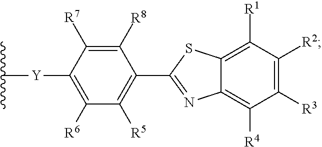

R.sup.1, R.sup.3, R.sup.4, R.sup.5, R.sup.6, R.sup.8, and R.sup.9 are hydrogen; and R.sup.2 is hydroxy.

In certain embodiments, the present invention relates to the aforementioned method and any of the attendant definitions, wherein the compound is represented by formula I; R.sup.7 is —N(H)—(CH.sub.2CHFCH.sub.2)-N(H)—R; R is

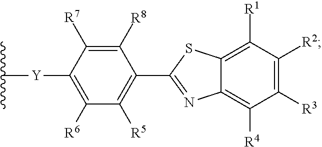

R.sup.1, R.sup.3, R.sup.4, R.sup.5, R.sup.6, R.sup.8, and R.sup.9 are hydrogen; and R.sup.2 is hydroxy.

In certain embodiments, the present invention relates to the aforementioned method and any of the attendant definitions, wherein the compound is represented by formula I; wherein R.sup.7 is —N(H)—(CH.sub.2CH.sup.18FCH.sub.2)-N(H)—R; R is

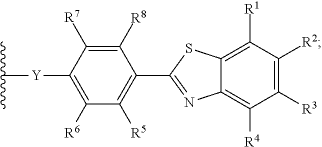

R.sup.1, R.sup.3, R.sup.4, R.sup.5, R.sup.6R.sup.8, and R.sup.9 are hydrogen; and R.sup.2 is hydroxy.

In certain embodiments, the present invention relates to the aforementioned method and any of the attendant definitions, wherein the compound is

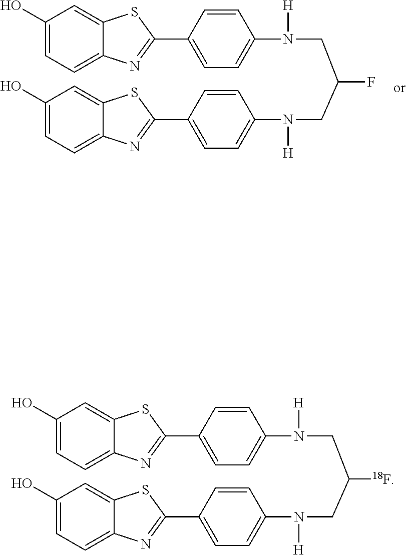

In certain embodiments, the present invention relates to the aforementioned method and any of the attendant definitions, wherein the pharmaceutical composition further comprises a sugar alcohol.

In certain embodiments, the present invention relates to the aforementioned method and any of the attendant definitions, wherein the pharmaceutical composition further comprises glycol, glycerol, erythritol, arabitol, xylitol, ribitol, mannitol, sorbitol, isomalt, maltitol, or lactitol.

In certain embodiments, the present invention relates to the aforementioned method and any of the attendant definitions, wherein the pharmaceutical composition further comprises mannitol.

### Definitions

Herein a number of terms are used extensively. The following definitions are provided to facilitate understanding of the invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 80 or fewer carbon atoms in its backbone (e.g., C.sub.1-C.sub.80 for straight chain, C.sub.3-C.sub.80 for branched chain), and alternatively, about 30 or fewer. Likewise, cycloalkyls have from about 3 to about 10 carbon atoms in their ring structure, and alternatively about 5, 6 or 7 carbons in the ring structure. As used herein, “fluoroalkyl” denotes an alkyl where one or more hydrogens have been replaced with fluorines; “perfluoroalkyl” denotes an alkyl where all the hydrogens have been replaced with fluorines.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “alkylene,” is art-recognized, and as used herein, pertains to a bidentate moiety obtained by removing two hydrogen atoms, either both from the same carbon atom, or one from each of two different carbon atoms, of a hydrocarbon compound, which may be aliphatic or alicyclic, or a combination thereof, and which may be saturated, partially unsaturated, or fully unsaturated. Examples of linear saturated C.sub.1-10alkylene groups include, but are not limited to, —(CH.sub.2).sub.n- where n is an integer from 1 to 10, for example, —CH.sub.2-(methylene), —CH.sub.2CH.sub.2-(ethylene), —CH.sub.2CH.sub.2CH.sub.2-(propylene), —CH.sub.2CH.sub.2CH.sub.2CH.sub.2-(butylene), —CH.sub.2CH.sub.2CH.sub.2CH.sub.2CH.sub.2-(pentylene) and —CH.sub.2CH.sub.2CH.sub.2CH.sub.2CH.sub.2CH.sub.2-(hexylene). Examples of branched saturated C.sub.1-10alkylene groups include, but are not limited to, —CH(CH.sub.3)-, —CH(CH.sub.3)CH.sub.2-, —CH(CH.sub.3)CH.sub.2CH.sub.2-, —CH(CH.sub.3)CH.sub.2CH.sub.2CH.sub.2-, —CH.sub.2CH(CH.sub.3)CH.sub.2-, —CH.sub.2CH(CH.sub.3)CH.sub.2CH.sub.2-, —CH(CH.sub.2CH.sub.3)-, —CH(CH.sub.2CH.sub.3)CH.sub.2-, and —CH.sub.2CH(CH.sub.2CH.sub.3)CH.sub.2-. Examples of linear partially unsaturated C.sub.1-10alkylene groups include, but are not limited to, —CH.dbd.CH-(vinylene), —CH.dbd.CH—CH.sub.2-, —CH.dbd.CH—CH.sub.2-CH.sub.2-, —CH.dbd.CH—CH.sub.2-CH.sub.2-CH.sub.2-, —CH.dbd.CH—CH.dbd.CH—, —CH.dbd.CH—CH.dbd.CH—CH.sub.2-, —CH.dbd.CH—CH.dbd.CH—CH.sub.2-CH.sub.2-, —CH.dbd.CH—CH.sub.2-CH.dbd.CH—, and —CH.dbd.CH—CH.sub.2-CH.sub.2-CH.dbd.CH—. Examples of branched partially unsaturated C.sub.1-10alkylene groups include, but are not limited to, —C(CH.sub.3).dbd.CH—, —C(CH.sub.3).dbd.CH—CH.sub.2-, and —CH.dbd.CH—CH(CH.sub.3)-. Examples of alicyclic saturated C.sub.1-10 alkylene groups include, but are not limited to, cyclopentylene (e.g., cyclopent-1,3-ylene), and cyclohexylene (e.g., cyclohex-1,4-ylene). Examples of alicyclic partially unsaturated C.sub.1-10alkylene groups include, but are not limited to, cyclopentenylene (e.g., 4-cyclopenten-1,3-ylene), and cyclohexenylene (e.g., 2-cyclohexen-1,4-ylene, 3-cyclohexen-1,2-ylene, and 2,5-cyclohexadien-1,4-ylene).

The term “haloalkylene,” as used herein, pertains to a bidentate alkylene moiety as described above wherein at least one hydrogen atom has been replaced by a halogen. The term “fluoroalkylene,” as used herein, pertains to a bidentate alkylene moiety as described above wherein at least one hydrogen atom has been replaced by a fluorine. The term “monofluoroalkylene,” as used herein, pertains to a bidentate alkylene moiety as described above wherein only one hydrogen atom has been replaced by a fluorine. The term “[F-18] fluoroalkylene,” as used herein, pertains to a bidentate alkylene moiety as described above wherein at least one hydrogen atom has been replaced by a F-18. The term “[F-18] monofluoroalkylene,” as used herein, pertains to a bidentate alkylene moiety as described above wherein only one hydrogen atom has been replaced by a F-18.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, aromatic or heteroaromatic moieties, trifluoromethyl, cyano, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkylthio, sulfonyl, ketone, aldehyde, ester, a heterocyclyl, an aromatic or heteroaromatic moiety, trifluoromethyl, cyano, or the like.

The team “nitro” is art-recognized and refers to —NO.sub.2; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO.sub.2.sup.-. “Halide” designates the corresponding anion of the halogens, and “pseudohalide” has the definition set forth on page 560 of “Advanced Inorganic Chemistry” by Cotton and Wilkinson, that is, for example, monovalent anionic groups sufficiently electronegative to exhibit a positive Hammett sigma value at least equaling that of a halide (e.g., CN, OCN, SCN, SeCN, TeCN, N.sub.3, and C(CN).sub.3).

The terms “amine” and “amino” are art-recognized and refer to both unsubstituted and substituted amines, e.g., a moiety that may be represented by the general formulas:

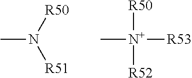

wherein R50, R51, R52 and R53 each independently represent a hydrogen, an alkyl, an alkenyl, —(CH.sub.2).sub.m-R61, or R50 and R51 or R52, taken together with the N atom to which they are attached complete a heterocycle having from 4 to 8 atoms in the ring structure; R61 represents an aryl, a cycloalkyl, a cycloalkenyl, a heterocycle or a polycyclic; and m is zero or an integer in the range of 1 to 8. In other embodiments, R50 and R51 (and optionally R52) each independently represent a hydrogen, an alkyl, an alkenyl, or —(CH.sub.2).sub.m-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

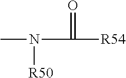

wherein R50 is as defined above, and R54 represents a hydrogen, an alkyl, an alkenyl or —(CH.sub.2).sub.m-R61, where m and R61 are as defined above.

The term “amido” is art recognized as an amino-substituted carbonyl and includes a moiety that may be represented by the general formula:

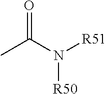

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH.sub.2).sub.m-R61, wherein m and R61 are defined above. Representative alkylthio groups include methylthio, ethyl thio, and the like.

The term “carboxyl” is art recognized and includes such moieties as may be represented by the general formulas:

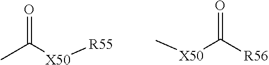

wherein X50 is a bond or represents an oxygen or a sulfur, and R55 and R56 represents a hydrogen, an alkyl, an alkenyl, —(CH.sub.2).sub.m-R61 or a pharmaceutically acceptable salt, R56 represents a hydrogen, an alkyl, an alkenyl or —(CH.sub.2).sub.m-R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiol carbonyl” group. Where X50 is a sulfur and R55 or R56 is not hydrogen, the formula represents a “thiol ester.” Where X50 is a sulfur and R55 is hydrogen, the formula represents a “thiol carboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C.dbd.O)NRR′, where R and R′ are independently H, aliphatic groups, aryl groups or heteroaryl groups.

The term “oxo” refers to a carbonyl oxygen (.dbd.O).

The terms “oxime” and “oxime ether” are art-recognized and refer to moieties that may be represented by the general formula:

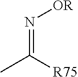

wherein R75 is hydrogen, alkyl, cycloalkyl, alkenyl, alkynyl, aryl, aralkyl, or —(CH.sub.2).sub.m-R61. The moiety is an “oxime” when R is H; and it is an “oxime ether” when R is alkyl, cycloalkyl, alkenyl, alkynyl, aryl, aralkyl, or —(CH.sub.2).sub.m-R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH.sub.2).sub.m-R61, where m and R61 are described above.

The term “sulfonate” is art recognized and refers to a moiety that may be represented by the general formula:

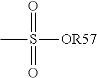

in which R57 is an electron pair, hydrogen, alkyl, cycloalkyl, or aryl.

The term “sulfate” is art recognized and includes a moiety that may be represented by the general formula:

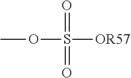

in which R57 is as defined above.

The term “sulfonamido” is art recognized and includes a moiety that may be represented by the general formula:

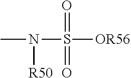

in which R50 and R56 are as defined above.

The term “sulfamoyl” is art-recognized and refers to a moiety that may be represented by the general formula:

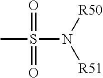

in which R50 and R51 are as defined above.

The term “sulfonyl” is art-recognized and refers to a moiety that may be represented by the general formula:

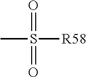

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

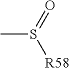

in which R58 is defined above.

The term “phosphoryl” is art-recognized and may in general be represented by the formula:

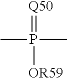

wherein Q50 represents S or O, and R59 represents hydrogen, a lower alkyl or an aryl. When used to substitute, e.g., an alkyl, the phosphoryl group of the phosphoryl alkyl may be represented by the general formulas:

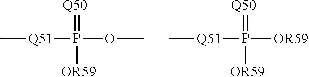

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

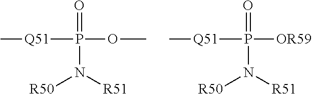

wherein Q51, R50, R51 and R59 are as defined above.

The term “phosphonamidite” is art-recognized and may be represented in the general formulas:

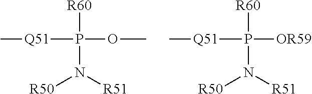

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH.sub.2).sub.m-R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methane sulfonate ester, and nonafluorobutanesulfonate ester functional groups and molecules that contain said groups, respectively.

The definition of each expression, e.g., alkyl, m, n, and the like, when it occurs more than once in any structure, is intended to be independent of its definition elsewhere in the same structure.

The abbreviations Me, Et, Ph, Tf, Nf, Ts, and Ms represent methyl, ethyl, phenyl, trifluoromethanesulfonyl, nonafluorobutanesulfonyl, p-toluene sulfonyl and methane sulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, “Handbook of Chemistry and Physics”, 67th Ed., 1986-87, inside cover.

As used herein, the term “subject” or “individual” refers to a human or other vertebrate animal. It is intended that the term encompass “patients.”

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition.

The term “conjugated” refers to ionically or covalently attached (e.g., via a crosslinking agent).

A “chelating structure” refers to any molecule or complex of molecules that bind to both label and targeting moiety. Examples include N.sub.2S.sub.2 structure, a HYNIC (hydrazinonicotinic acid) group-containing structure, a 2-methylthiolnicotinic acid group containing structure, a carboxylate group-containing structure and the like.

A “radioimaging agent” refers to a composition capable of generating a detectable image upon binding with a target and shall include radionuclides such as, for example, sup.18F, sup.76Br, sup.77Br, sup.123I, sup.124I, sup.125I, .sup.99mTc, sup.68Cu, .sup.64Cu and sup.68Ga.

A “fluorescence imaging agent” refers to a composition capable of generating a detectable optical imaging upon binding with a target with or without specific wave length of light activation and shall include fluorophores. The preferred fluorescence agents are near infra-red light absorbing agents.

A “target” refers to an in vivo site to which imaging compounds binds. A preferred target is a brain tissue from a subject suffering from AD or an amyloidosis-associated pathological condition. A “targeting molecule” is any molecule or biological entity that specifically accumulates in brain tissue from a subject suffering from AD or an amyloidosis-associated pathological condition.

As used herein, “pharmaceutically-acceptable carrier” includes any and all solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds of the present invention. Examples of such carriers include oils such as corn oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The formulation used in the present invention may also contain stabilizers, preservatives, buffers, antioxidants, or other additives known to those of skill in the art. The use of such media and agents for pharmaceutically-active substances is well known in the art. Supplementary active compounds can also be incorporated into the imaging agent of the invention. The imaging agent of the invention may further be administered to an individual in an appropriate diluent or adjuvant, co-administered with enzyme inhibitors or in an appropriate carrier such as human serum albumin or liposomes. Pharmaceutically-acceptable diluents include sterile saline and other aqueous buffer solutions. Adjuvants contemplated herein include resorcinols, non-ionic surfactants such as polyoxyethylene oleyl ether and n-hexadecyl polyethylene ether. Enzyme inhibitors include pancreatic trypsin inhibitor, diethylpyrocarbonate, and trasylol. Liposomes inhibitors include water-in-oil-in-water CGF emulsions, as well as conventional liposomes (see J. Neuroimmunol. 1984, 7, 27).

A “sugar alcohol” (also known as a polyol, polyhydric alcohol, or polyalcohol), as used herein, is a hydrogenated form of carbohydrate, whose carbonyl group (aldehyde or ketone, reducing sugar) has been reduced to a primary or secondary hydroxyl group. Examples of sugar alcohols include glycol, glycerol, erythritol, arabitol, xylitol, ribitol, mannitol, sorbitol, isomalt, maltitol, and lactitol.

As described herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. See J. Pharm. Sci. 1977, 66, 1-19.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluene sulfonic, methane sulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases.

The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, J. Pham. Sci. 1977, supra).

Preferably, the imaging agent of the present invention is administered intravenously, and the imaging agent will be formulated as a sterile, pyrogen-free, parenterally-acceptable aqueous solution. The preparation of such parenterally-acceptable solutions, having due regard to pH, isotonicity, stability, and the like, is within the skill in the art. A preferred formulation for intravenous injection should contain, in addition to the imaging agent, an isotonic vehicle such as Sodium Chloride Injection, Ringer's Injection, Dextrose Injection, Dextrose and Sodium Chloride Injection, Lactated Ringer's Injection, or another vehicle as known in the art.

The amount of imaging agent used for diagnostic purposes and the duration of the imaging study will depend upon the nature and severity of the condition being treated, on the nature of therapeutic treatments which the patient has undergone, and on the idiosyncratic responses of the patient. Ultimately, the attending physician will decide the amount of imaging agent to administer to each individual patient and the duration of the imaging study.

The diagnostic imaging amounts are preferably about 3 to 15 millicuries (mCi) for a 70-kg normal adult, more preferably being about 1-25 mCi for a 70-kg normal adult.

The ultimate solution form is preferably sterile. Sterilization can be accomplished by any art recognized technique, including but not limited to, addition of antibacterial of antifungal agents, for example, paraben, chlorobutanol, phenol, sorbic acid, thimerosal, and the like.

More specifically, the compounds that can be formulated into a pharmaceutical composition include a therapeutically-effective amount of the compound and a pharmaceutically-acceptable carrier. The therapeutically-effective amount of the compound and the specific pharmaceutically-acceptable carrier will vary depending upon, e.g., the age, weight, sex of the subject, the mode of administration, and the type of viral condition being treated.

In an aspect, the pharmaceutical composition which can be used includes the compounds of the present invention in effective unit dosage form. As used herein, the term “effective unit dosage” or “effective unit dose” is used herein to mean a predetermined amount sufficient to be effective against AD or the like. Examples include amounts that enable detecting and imaging of amyloid deposit(s) in vivo or in vitro, that yield acceptable toxicity and bioavailability levels for pharmaceutical use, and/or prevent cell degeneration and toxicity associated with fibril formation.

The pharmaceutical compositions may contain the compound used in the method of this invention in an amount of from 0.01 to 99% by weight of the total composition, preferably 0.1 to 80% by weight of the total composition. For intravenous injection, the dose may be about 0.1 to about 30 mg/kg/day, preferably about 0.5 to about 10 mg/kg/day. Fluorescence agents will be administered in several mug/kg to several mg/kg. For example, 1-10 mg/kg.

When the compounds according to the invention are formulated for injection, the dose may be presented in unit dose form in ampoules or in multi-dose containers with added pharmaceutically-acceptable adjuvants such as a preservative.

In addition, the compositions may take forms such as suspensions, solutions, or emulsions in oily or aqueous vehicles, and may contain formulating agents, such as suspending, stabilizing, or dispersing agents, isotonic agents and/or dissolving co-solvents conventionally cited in the pharmaceutical art.

For systemic administration, the daily dosage as employed for adult human treatment will range from about 0.1 mg/kg to about 150 mg/kg, preferably about 0.2 mg/kg to about 80 mg/kg.

Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds of the present invention. Examples of such carriers include oils such as corn oil, buffers such as PBS, saline, polyethylene glycol, glycerin, polypropylene glycol, dimethyl sulfoxide, an amide such as dimethylacetamide, a protein such as albumin, and a detergent, such as Tween 80, mono-, oligopoly saccharides, such as glucose, lactose, cyclodextrins and starch.

The pharmaceutical compositions may contain other active ingredients, such as antimicrobial agents and other adjuvants such as benzyl alcohol and phenol compounds and diluents conventionally used in the art.

It should be understood that the embodiments and examples described herein are given by way of illustration, not limitation. Various changes and modifications within the scope of the present invention will become apparent to those skilled in the art from the present description.

### EXEMPLIFICATION

It should be understood that the above-described embodiments and the following examples are given by way of illustration, not limitation. Various changes and modifications within the scope of the present invention will become apparent to those skilled in the art from the present description.

### Example 1

### Synthesis of [N-2[18F] fluoropropyl]-2-(4′-(methylamino) phenyl)-6-hydroxythiazole

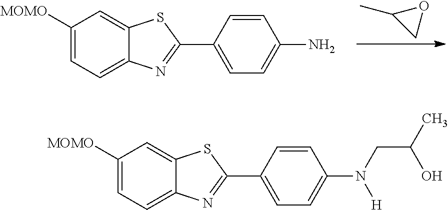

2-(4′-Aminophenyl)-6-methoxymethoxybenzothiazole (6-MOMO-BTA-O) was prepared according to a known procedure. See Mathis, C. A. et. al., J. Med. Chem. 2003, 46, 2470-2754. 6-MOMO-BTA-O (500 mg, 1.7 mmol), propylene oxide (500 mg, 8.5 mmol) and NaH (8 1 mg, 2 mmol, 60% oil dispersion) were heated at 100° C. in acetonitrile (30 mL) for 4 hr. The reaction mixture was poured over ice water (50 mL) and extracted with ether (3×20 mL). The combined extracts were dried (Na.sub.2S0.sub.4) and solvent was evaporated. Chromatography on silica gel using 50/50 ethyl acetate hexane gave 147 mg (43%) of the isopropanol derivative.

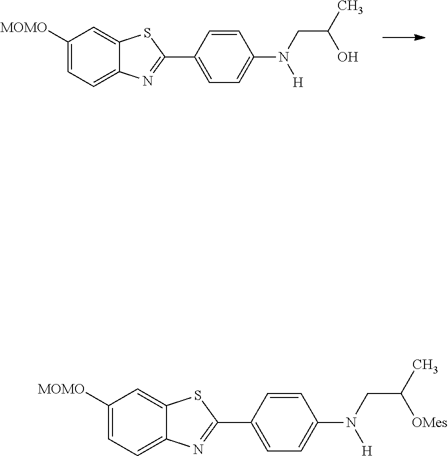

[N-2-propanol]-2-(4′-methylaminophenyl)-6-methoxymethoxybenzothiazole (100 mg, 0.29 mmol) and pyridine (0.5 mL) in methylene chloride (20 mL) was treated with methane sulfonyl chloride (0.66 mg, 58 mmol) for 4 hr. The mixture was washed with saturated NaHCO.sub.3 (30 mL) and the organic layer was dried.

After removal of volatiles by vacuum, the crude oil was chromatographed on silica gel using methylene chloridel methanol (95:5) to give 50 mg (41%) of the mesylate.

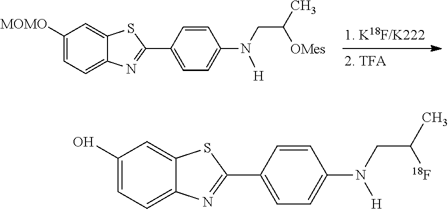

The mesylate (5 mg) in acetonitrile (1.00 mL) was added to a sealed vial containing dried K.sup.18F/kryptofix (100 mCi) and heated at 120.degree. C. for 10 min Once cooled, the mixture was purified on a Silica SepPak using 10% methanol in methylene chloride. After solvent removal, the intermediate was treated with TFA at 100.degree. C. for 10 min and solvent was removed by a nitrogen stream. The F-18 labeled derivative was dissolved in PBS/acetonitrile and purified on a C-18 column. [N-2[18F] fluoropropyl]-2-(4′-(methylamino) phenyl)-6-hydroxythiazole (10 mCi) was prepared within 90 min with 98% radiochemical purity.

See also the “Synthesis Examples” in International Application No. PCT/US2005/023618, hereby incorporated by reference.

### Example 2

### Synthesis of a Dimer of [N-2[18F] fluoropropyl]-2-(4′-(methylamino) phenyl)-6-hydroxythiazole

The dimer series relating to the structure of [N-2[18F] fluoropropyl]-2-(4′-(methylamino) phenyl)-6-hydroxythiazole (F-18 MPHT) can be synthesized.

Alternatively, the synthesis of a 2-[F18] fluoropropane dimer of 2-(4′-aminophenyl)-6-methoxymethoxybenzothiazole can be prepared as outlined below.

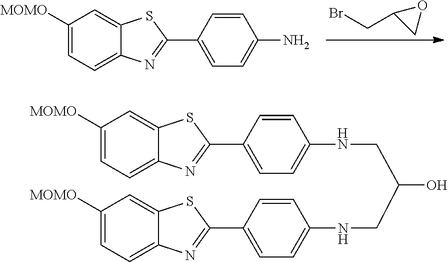

First, epibromohyrin and 2-(4′-aminophenyl)-6-methoxymethoxybenzothiazole is heated in acetonitrile and in the presence of lithium bromide (cat.) at 150.degree. C. for 12 hr.

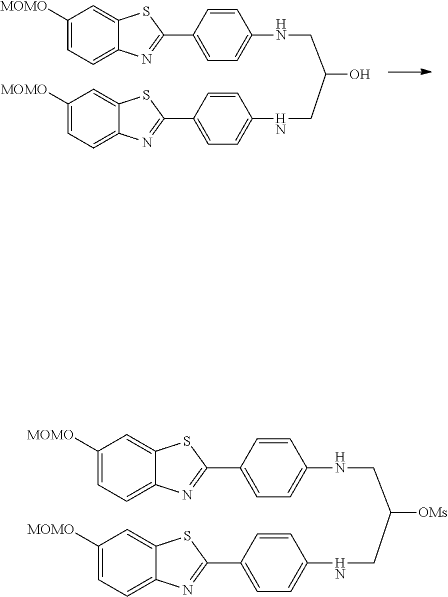

The resulting alcohol in pyridine and methylene chloride is then treated with methane sulfonyl chloride for 4 hr. The mixture is washed with saturated NaHCO.sub.3 (30 mL) and the organic layer dried. After removal of volatiles by vacuum, the crude oil is chromatographed on silica gel using methylene chloride/methanol (95:5) to give the mesylate.

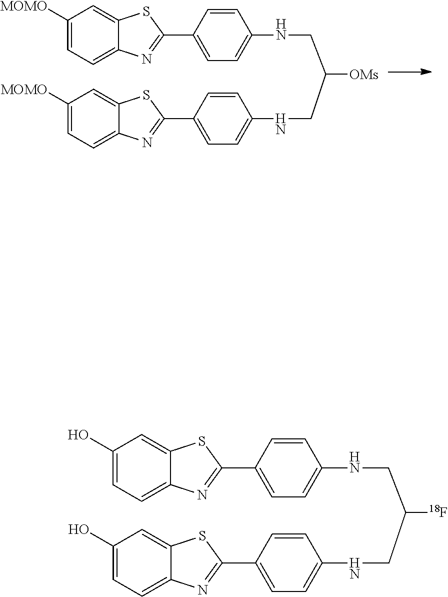

The mesylate (5 mg) in acetonitrile (100 muL) is added to a sealed vial containing dried K.sup.18F/kryptofix (100 mCi) and heated at 120 degree ° C. for 10 min. Once cooled, the mixture is purified on a Silica SepPak using 10% methanol in methylene chloride. After solvent removal, the intermediate was treated with TFA at 100° C. for 10 min and solvent is removed by a nitrogen stream. The F-18 labeled derivative is dissolved in PBS/acetonitrile and purified on a C-18 column.

### Technical Field of the Invention

The present invention relates to a method for the synthesis of ¹⁸F-labelled compounds and in particular ¹⁸F-labelled compounds that are useful as positron emission tomography (PET) tracers.

### Description of Related Art

Leukemia is a cancer that begins in the bone marrow (the soft inner part of some bones), but in most cases, moves into the blood. It can then spread to other parts of the body, such as organs and tissues. Once these white blood cells are affected by leukemia, they do not go through their normal process of maturing.

The lymphocytes continue to reproduce, build up and invade the blood quickly. ALL is an aggressive type of leukemia; without treatment, most patients with acute leukemia would live only a few months. ¹⁸F-FLT, a thymidine analogue labeled with an 18F isotope, was introduced in 1998 by Shields et al and ¹⁸F-FLT PET is used to study cell proliferation in vivo.

The radioisotope suitable for detection in positron emission tomography (PET) have notably short half-lives. Fluorine-18 (¹⁸F) has a half-life of about 110 minutes. Synthetic methods for the production of compounds labelled with these radionuclides need to be as quick and as high yielding as possible. This is particularly important in the case of compounds destined to be used for in vivo imaging, commonly known as PET tracers. Furthermore, the step of adding the radioisotope to the compound should be as late as possible in the synthesis, and any steps taken following the addition of radioisotope for the work up and purification of the radioisotope-labelled compounds should be completed with as little time and effort as possible.

Taking [¹⁸F]FLT, Brasse et al. (2016 Nuc Med Biol) describes an automated method for its synthesis.

### 2. Materials and Methods

### 2.1 Chemicals, Reagents and Apparatus

Brasse et al. report that (5′-O-DMTr-2′-deoxy-3′-O-nosyl-D-threo-pentofuranosyl)-3-N-BOC-thymine, stavudine (d4T,2′-3′-didehydro-2′-3′-dideoxythymidine) and 3′-deoxy-3′-fluoro-thymidine (FLT) were purchased from ABX. K2CO3 99.99% and Kryptofix K2.2.2 (4,7,13,16,21,24-hexaoxa-1,10-diazabicyclo-[8.8.8]-hexacosane) were purchased from Aldrich. Accell plus QMA carbonate light cartridges were obtained from ABX or Waters and used as received. Alumina N cartridges (Alox N, medium size) were purchased from Macherey-Nagel and washed with 2×5 mL of pure water then dried thoroughly before use. [18O] H2O ([18O]>97%) was purchased to Sercon. Pure H20 (18.2 MΩ) was produced with a Purelab option Q purification system (Veolia®). Anhydrous acetonitrile>99.8%, 3-methyl-pentan-3-ol 99%, ethanol (absolute, HPLC grade), methanol (HPLC grade), ethyl acetate, hydrochloric acid, NaOH and sodium acetate were purchased from Aldrich and used as received. Sodium chloride 0.9% sterile solution was purchased from B BRAUN medical.

Thin layer chromatography was performed on aluminum back coated TLC silica gel 60F254 plates from Millipore. (Id.)

HPLC Dionex® U3000 equipped with a DAD detector, a radioactivity detector and a syncronis 250×4.6 mm (5 μm) analytical column was used for quality control and specific activity determination. (Id.)

Radio TLC reader miniGita (Raytest®) was used to determine ¹⁸F incorporation in manual fluorination reactions. (Id.)

Well counter: dose calibrator ISOMED 2010 was purchased from Raytest. (Id.)

Dry block heater: reacti-therm from Thermo Fisher Scientific® with aluminum block and reacti-vap evaporator connected to nitrogen inlet were used for water evaporation and to carry out manual fluorination reaction.

### 2.2 Manual Synthesis

Brasse et al. further report that manual syntheses were performed in 5 mL V-vials fitted with hole caps and teflon coated septa. A sample of 18F in water (10-50 MBq) was transferred in a 1 mL syringe, counted in well counter and passed through a QMA cartridge. The cartridge was dried with 5 mL of air and the radioactivity was eluted with potassium carbonate and K222 kryptofix (acetonitrile/water) in a V-vial and counted for determination of eluted activity.

The azeotropic solution obtained by iterative addition of acetonitrile (3×1 mL) was evaporated in a dry block heater at 100° C. under nitrogen flow. The precursor (5 or 10 mg, solubilized in 750 μL of 3-methyl-pentan-3-ol and 250 μL of acetonitrile) was added and the sealed vial was heated for 15 minutes at 110° C. Samples (150 μL) were withdrawn at 5, 10 and 15 minutes, diluted with methanol and water (50 μL each) and spotted on TLC (eluted with ethyl acetate) for 18F incorporation determination using a minigita radio-TLC reader. (Id.)

### 2.3 Automated Synthesizer

Brasse et al. further provide that a Raytest R&D synchrom® dual reactor was used for the radiosynthesis with minor modifications. Only one reactor is used for the synthesis, Al₂O₃ neutral cartridge was inserted between valve C1 and D4, outlet of the pump and vents were connected to gas bags to avoid any radioactive releases in the hot cell ventilation system, 3 additional valves (G3-G5) and a 20-mL vial were added to measure the ¹⁸F activity transferred from the target. The system is equipped with a semi preparative HPLC (Knauer) including an isocratic pump, a 254-nm fixed wavelength UV detector, a radioactivity detector and a 5-mL stainless steel injection loop. Purification was done on a syncronis 250×10 mm (5 μm) semi-preparative column at 3 mL/min.

### 2.4 Step 1: Nucleophilic Fluorination

i) [18F] fluorine from target vial transferred through a QMA cartridge on the module and water recovered in 10 mL V-vial.

ii) [18F] fluorine eluted into reactor 1 with 12-15 mg of K222, 0.59 mg of K2CO3 in 800 μL of acetonitrile and 380 μL of water (SC1).

iii) [18F] KF dried under reduced pressure and Argon flow at 90° C. with successive addition of acetonitrile (SC2, 1.7 mL).

iv) The precursor 3-N-Boc-5′-O-dimethoxytrityl-3′-O-nosyl-thymidine (5 mg) dissolved in 250 μL of acetonitrile and 750 μL of 3-methyl-pentan-3-ol (SC3) is added into the reactor.

An ACSI 24 MeV® cyclotron was used for 18F production on a small volume target. (Id.) Irradiation of [18O] H2O (1 mL) was performed at 16.5 MeV, 30 μA during 15 minutes to produce a typical dose of 19 GBq (EOB). After 5 minutes of cooling the radioactivity is transferred to the hot cell under He pressure and the target is rinsed twice (2×1 mL) with pure water. Total transferred activity is counted (well counter) and sent into the Raytest module under the pressure. Residual activity in the intermediate vial after transfer is counted. Starting from 19 GBq (EOB), 16 GBq were transferred to the synthesizer for [18F] FLT synthesis. (Id.)

### 2.5 Quality Control and Specific Activity Determination for Preclinical Use

Brasse et al. further report that purified [18F] FLT was injected on analytical HPLC (25 μL) for determination of chemical and radiochemical purities. A second injection with the non-radioactive authentic reference FLT (3′-deoxy-3′-fluoro-thymidine) was done to assay the identity of the radioactive compound. The column was eluted with a gradient of acetonitrile in water (H2O/CH3CN 90/10 to 80/20 over 10 min then 80/20 to 70/30 over 15 min.) at 0.7 mL/min and UV detection was performed at 265 nm.

The specific activity was determined by injection on analytical HPLC of a known radioactive dose of [¹⁸F] FLT (0.7 to 1.5 MBq) and quantification of the amount of FLT by reporting the UV signal area on a calibration curve (R²=0.999). (Id.)

The pH of the solution was determined by applying a drop of the purified solution onto a pH indicator strip and comparing the result with the provided scale. The volumic activity (MBq/mL) at the end of synthesis was determined by measuring the activity of a known volume of purified [¹⁸F] FLT (25 to 50 μL). (Id.)

### 3.1 Manual Synthesis, Determination of 18F Incorporation

The following step of the evaluation, as reported by Brasse et al., was to perform the radiofluorination of precursor VII under various conditions (amount of precursor and base). The solutions eluted from QMA cartridges (see 3.1) were evaporated under N2 flow at 100° C. with iterative addition of CH3CN (3×1 mL). After drying, the precursor 5 or 10 mg in 1 mL of solvent (3-methyl-pentan-3-ol/CH3CN 75/25) was added and the vial was heated for 15 minutes at 110° C. Samples were withdrawn at 5, 10, 15 minutes, diluted and analyzed by radio-TLC (eluted with 100% ethyl acetate). The precursor to base ratio (P/B) was critical to achieve high incorporation of the 18F fluorine. No significant difference in conversion was observed between 10 and 15 minutes. Better results were obtained when the precursor to base ratio was superior to 1.3; using such conditions 97% and 93% conversion were observed using 10 mg and 5 mg of precursor respectively. Although these results represent the higher incorporation percentages reported so far, it is worth to note that they reflect only the composition of the liquid phase, the radioactivity in solution usually represents 90-95% of the initial amount introduced in the vial (part of it get stuck on the glass wall of the V-vial). (Id.)

Brasse et al. indicates that they were not able to identify a parameter that could influence positively or negatively this amount of lost radioactivity. One reason might come from the manual drying step that led to some fluctuating binding of 18F fluorine on the glassware. (Id.)

### 3.2 Automated Synthesis

Brasse et al. report that the above-mentioned results prompted them to adapt the manual synthesis on the Raytest automate. They first adapted the conditions using 10 mg of precursor along with 1 mg of K₂CO₃. As the reactor of the synthesizer is suitable for reaction volumes of 1 mL no dilution was necessary and the concentration of the precursor could be kept the same as in manual synthesis. Classical automated sequence was used to transfer the radioactivity into the reactor and to dry the [¹⁸F]-KF.

The temperature of the reactor was raised to 110° C. before addition of the precursor dissolved in acetonitrile and 3-metyl-pentan-3-ol. Hindered alcohols are now often used in FLT synthesis to reduce the formation of by-products especially when they are generated by the competitive elimination reaction [32]. Among the potential hindered alcohols, 3-methyl-pentan-3-ol was chosen for its 123° C. boiling point; in such conditions the reaction can be performed at high temperature without generating an excessive pressure in the reactor. During the synthesis, the maximum pressure observed never exceeded 230 KPa (2.3 bars). (Id.)

The other advantage is the easy elimination of the alcohol under reduced pressure, this evaporation step is mandatory as otherwise the deprotection step using 1M aqueous HCl fails due to the non-miscibility of the reagents (deprotection using other acid, ie CF₃CO₂H was not attempted). (Id.)

Brasse et al. report that the fluorinations were carried out at 110-112° C. during 15 minutes and after evaporation 250 μL of acetonitrile were added to the reactor to facilitate the solubilization of the intermediate during the deprotection step. In the classical set-up, CH₃CN and HCl are located in separated solvent containers (SC4 and SC5) but a mixture of CH₃CN and aqueous HCl can also be used in a single solvent container. Addition of a larger volume of CH₃CN considerably impaired the final HPLC purification. (Id.)

Deprotection is carried out at 90° C. during 10 minutes, the reactor is cooled down and the reaction is neutralized using NaOH 1N with 0.25 M CH₃CO₂Na. The crude product is then passed through an alumina neutral cartridge and directly sent into the HPLC loop for semi-preparative purification on a syncronis column. (Id.)

According to the process reported by Brasse et al., pure [¹⁸F] FLT was collected at 24 minutes (chromatogram of semi-preparative HPLC) after injection and ready to use (already formulated in NaCl 0.9% with 8% ethanol), the average yield (decay corrected) was 56% (±5%, n=5). Starting from 16 GBq they obtained a ready to inject solution of 7.5 mL with a [¹⁸F] FLT concentration greater than 500 MBq/mL. The synthesis time was 52 minutes without purification and 81 minutes from start of synthesis to the final collect of the purified product (HPLC included) (Id.)

With 5 mg of precursor the automated synthesis required only minor modifications; the QMA cartridge is eluted with a solution containing 12-14 mg of K222, 0.59 mg of K₂CO₃ in 800 μL of CH₃CN and 380 μL of H₂O. As the amount of water eluted from QMA is more important, 1 minute of drying is added to the sequence. The remaining of the synthesis remained unchanged. Using such a low amount of precursor, [¹⁸F] FLT was obtained after purification in 54% (n=4, min. 45% max. 60%) decay corrected yield. (Id.)

Brasse et al. report that only few percent of yield difference were observed during our synthesis when 5 mg were used instead of 10 mg. When compared to recently published radiosynthesis using 10 mg or even 5 mg of precursor our automated synthesis achieved high yields of pure [¹⁸F] FLT. (Id.)

### 3.3 Quality Control and Specific Activity

Brasse et al. further report that a quality control for pre-clinical use was performed. The results demonstrated a chemical and radiochemical purity above 95% and co-injection of authentic FLT proved the identity of the produced [¹⁸F] FLT (Rₜ=12.4 min). (Id.)

The pH of the solution ranged from 6 to 7 and the volumic activity was superior to 500 MBq/mL for each batch (n=9) with a specific activity of 35 to 72 GBq/μmole. (Id.)

No stavudine could be detected by HPLC in the final product (Rₜ=9.1 min for the stavudine under same analytical conditions). (Id.)

### Summary of the Invention—Method for Preparing an ¹⁸F-Labelled Compound

The present invention provides an improved method to prepare an ¹⁸F-labelled compound where the synthesis comprises a hydrolytic deprotection step. Specifically, the method of the invention permits neutralization of an acidic or basic crude product without using any neutralizing chemicals. Instead, the product is trapped on an SPE column and then thoroughly rinsed with water. As a consequence of this process simplification, the method of the invention can more readily be carried out on an automated synthesizer. In addition to the radiofluorination method of the invention, the present invention provides a cassette designed to carry out the method on an automated synthesizer.

### Detailed Description of Preferred Embodiments

The present invention therefore provides in one aspect a method comprising:

(i) labelling a protected precursor compound with ¹⁸F;

(ii) deprotecting the ¹⁸F-labelled compound obtained in step (i) by hydrolysis;

(iii) diluting the deprotected ¹⁸F-labelled compound obtained in step (ii) with water;

(iv) trapping the deprotected ¹⁸F-labelled compound on a solid-phase extraction (SPE) column by passing the diluted solution obtained in step (iii) through said column;

(v) eluting the deprotected ¹⁸F-labelled compound obtained in step (iv) from the SPE column; with the proviso that no neutralizing step is carried out following the deprotection step. An “¹⁸F-labelled compound” in the context of the present invention is a chemical compound comprising at least one ¹⁸F atom. Preferably, an ¹⁸F-labelled compound of the present invention comprises only one ¹⁸F atom.

The term “labelling” in the context of the present invention refers to the radiochemical steps involved to add ¹⁸F to a compound. The precursor compound is reacted with a suitable source of ¹⁸F to result in the ¹⁸F-labelled compound. A “suitable source of ¹⁸F” is typically either ¹⁸F-fluoride or an ¹⁸F-labelled synthon. ¹⁸F-fluoride is normally obtained as an aqueous solution from the nuclear reaction ¹⁸0(p,n)¹⁸F. In order to increase its reactivity and to avoid hydroxylated by-products resulting from the presence of water, water is typically removed from ¹⁸F-fluoride prior to the reaction, and fluorination reactions are carried out using anhydrous reaction solvents (Aigbirhio et al 1995 J Fluor Chem; 70: 279-)87). The removal of water from ¹⁸F-fluoride is referred to as making “naked” ¹⁸F-fluoride. A further step that is used to improve the reactivity of ¹⁸F-fluoride for radiofluorination reactions is to add a cationic counterion prior to the removal of water. Suitably, the counterion should possess sufficient solubility within the anhydrous reaction solvent to maintain the solubility of the ¹⁸F-fluoride. Therefore, counterions that are typically used include large but soft metal ions such as rubidium or cesium, potassium complexed with a cryptand such as Kryptofix™, or tetraalkylammonium salts, wherein potassium complexed with a cryptand such as Kryptofix™, or tetraalkylammonium salts are preferred.

The term “precursor” refers to a compound that when reacted with a suitable source of ¹⁸F results in the desired ¹⁸F-labelled compound.

When ¹⁸F-FLT is the ¹⁸F-labelled compound obtained by the method of the present invention, a preferred protected precursor compound is a compound of Formula I:

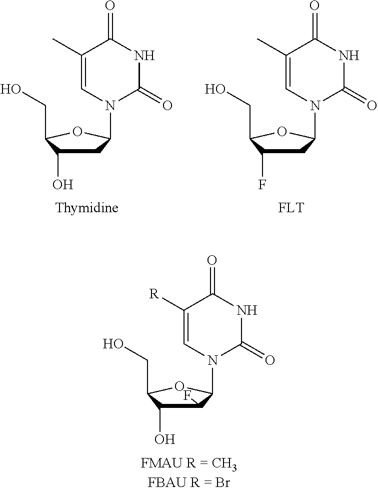

wherein:

R is a protecting group for the hydroxyl function; and,

R is preferably selected from CI, Br, I, tosylate (OTs), mesylate (OMs) and inflate (OTf), most preferably selected from OTs, OMs and OTf, and is most especially preferably OTs.

A most preferred precursor compound for the synthesis of 3′-Deoxy-3′-[¹⁸F]fluorothymidine is (2′-Deoxy-2′-fluoro-β-L-arabinofuranosyl)-5-methyluracil (FMAU); 3′-deoxy-3′-fluorothymidine (FLT); and/or 1-(2′-Deoxy-2′-[18F]fluoro-1-β-D-Arabinofuranosyl)-5-Bromouracil (FBAU)

In a preferred embodiment of the invention, the diluting step comprises:

(a) adding a first volume of water to said deprotected ¹⁸F-labelled compound to obtain a first diluted solution, and,

(b) adding subsequent volumes of water to aliquots of said first diluted solution to obtain subsequent diluted solutions.

It is intended that the diluting step will result in a reaction mixture having a polarity suitable to permit high and reproducible trapping on an apolar SPE column. Ideally, the diluted reaction mixture should not have more than around 10-15% organic solvent in water in order to achieve this aim. Aliquots of the diluted solution are passed through the SPE column so as to trap the deprotected ¹⁸F-labelled compound onto the column. Optionally, once all the diluted solutions has been passed through the SPE column, an additional step of washing the column with water may be carried out prior to the eluting step.

Preferably, the eluting step is carried out using a solution of aqueous ethanol. In the case of ¹⁸F-FLT, it is preferred that the eluting step is carried out with an aqueous ethanol solution comprising 2-20% ethanol, most preferably 5-10% ethanol. The sorbent of the SPE column for the present invention can be any silica- or polymeric-based apolar sorbent. Non-limiting examples of suitable apolar SPE columns include polymer-based Oasis HLB or Strata X SPE columns, or silica-based C2, C4, C8, CI 8, tC18 or C30 SPE columns. The SPE column of the invention is preferably selected from Oasis HLB, tCl 8, and Strata X. ¹⁸F-labelled PET tracers are now often conveniently prepared on an automated radiosynthesis apparatus. Therefore, in a preferred embodiment, the method of the present invention is an automated synthesis. The term “automated synthesis” refers to a chemical synthesis that is performed without human intervention. In other words, it refers to a process that is driven and controlled by at least one machine and that is completed without the need of manual interference.

The term “diluting” is well-known in the art and refers to the process of reducing the concentration of a solute in solution by mixing with more solvent. In the context of the present invention the solvent used in the diluting step is water. The purpose of the diluting step is to increase the polarity of the reaction mixture in order to permit high and reproducible trapping of the product on an apolar (also commonly termed “reverse-phase”) SPE column.

The term “trapping” in the present invention refers to the retention of the deprotected ^{t8}F-labelled compound on the SPE column by interactions between the deprotected ¹⁸F-labelled compound and the sorbent of the SPE column. These interactions are solvent-dependent.

The term “solid-phase extraction” (SPE) refers to the chemical separation technique that uses the affinity of solutes dissolved or suspended in a liquid (known as the mobile phase) for a solid through which the sample is passed (known as the stationary phase or sorbent) to separate a mixture into desired and undesired components. The result is that either the desired analytes of interest or undesired impurities in the sample are retained on the sorbent, i.e. the trapping step as defined above. The portion that passes through the sorbent is collected or discarded, depending on whether it contains the desired analytes or undesired impurities. If the portion retained on the sorbent includes the desired analytes, they can then be removed from the sorbent for collection in an additional step, in which the sorbent is rinsed with an appropriate eluent. The sorbent is typically packed between two porous media layers within an elongate cartridge body to form the “solid-phase extraction (SPE) column”. High-performance liquid chromatography (HPLC) is specifically excluded from the definition of SPE in the context of the present invention.

The term “neutralizing” as used herein refers to the process of adjusting the pH of a solution to bring it back to pH 7, or as close as possible to pH 7. Therefore, an acidic solution can be neutralized by adding a suitable amount of an alkali such as NaOH, and an alkaline solution can be neutralized by adding a suitable amount of an acid such as HCl.

The term “eluting” refers to the process of removing the desired compound from the SPE column by passing a suitable solvent through the column. The suitable solvent for eluting is one in which the interactions between the sorbent of the SPE column and the desired compound are broken thereby allowing the compound to pass through the column and be collected.

In the method of the present invention, a distinct neutralization step is not carried out. Rather, the step of diluting serves both to bring the pH to neutrality and to prepare the reaction mixture for SPE purification. As compared to the prior art methods, the method of the present invention is therefore simplified by removal of the neutralization step, which makes the method more straightforward to carry out and to automate.

The method of the invention may be applied to the synthesis of any ¹⁸F-labelled PET tracer that comprises ¹⁸F labelling of a precursor compound that comprises protecting groups and subsequent removal of the protecting groups by acid or alkaline hydrolysis. Non-limiting examples of such ¹⁸F-labelled PET tracer include ¹⁸F-fluorodeoxyglucose (¹⁸F-FDG), 6-[¹⁸F]-L-fluorodopa (¹⁸F-FDOPA), ¹⁸F-fluoro thymidine (¹⁸F-FLT), 1-H-1-(3-[¹⁸F]fluoro-2-hydroxypropyl)-2-nitroimidazole(¹⁸F-FMISO), ¹⁸F-1-(5-fluoro-5-deoxy-a- arabinofuanosyl)-2-mitroimidazole (¹⁸F-FAZA), 16-a-[¹⁸F]-fluoroestradiol (¹⁸F-FES), and 6-[′⁸F]-fluorometarminol (¹⁸F-FMR). Said ¹⁸F-labelled compound is preferably ¹⁸F-fluorodeoxyglucose (¹⁸F-FDG), 6-[¹⁸F]-L-fluorodopa (¹⁸F-FDOPA), ¹⁸F-fluorothymidine (F-FLT), or F-fluoromisonidazole (F-FMISO), and most preferably ¹⁸F-fluorothymidine (¹⁸F-FLT) or ¹⁸F-fluoromisonidazole (¹⁸F-FMISO).

The known synthesis of each of these PET tracers includes a deprotection step and a neutralization step (see for example chapters 6 and 9 of “Handbook of Radiopharmaceuticals” 2003; Wiley: by Welch and Redvanly, and chapter 8 of “Basics of PET Imaging, 2^{nd} Edition” 2010; Springer: by Saha). The method of the invention is carried out to obtain any of these PET tracers in purified Run in a straightforward manner by omitting the neutralization step and carrying out the diluting, trapping and eluting steps as defined herein. Examples of PET tracers which may be synthesized by the method of this aspect of the present invention include [¹⁸F]-fluorodeoxyglucose ([¹⁸F]-FDG), [¹⁸F]-fluorodihydroxyphenylalanine ([¹⁸F]-F-DOPA), [¹⁸F]-fluorouracil, [¹⁸F]-1-amino-3-fluorocyclobutane-1-carboxylic acid ([¹⁸F]-FACBC), [′⁸F]-altanserine, [¹⁸F]-fluorodopamine, 3′-deoxy-3′-¹⁸F-fluorothymidine [¹⁸F-FLT] and [¹⁸F]-fluorobenzothiazoles.

The structures of various ¹⁸F-labelled protected precursor compounds obtained in step (i) of the method of the present invention are as follows (wherein P¹ to P⁴ are each independently hydrogen or a protecting group):

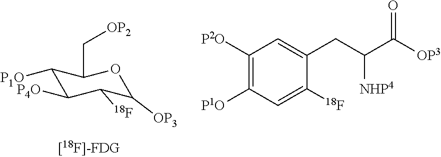

In one embodiment, the method of the invention is used for the synthesis of 18F-FLT:

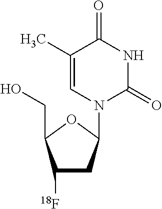 [SMILES: Cc1cn([C@H]2C[C@@H]([18F])[C@@H](CO)O2)c(=O)[nH]c1=O]

There are several commercially-available examples of such apparatus, including Tracerlab™ and Fastlab™ (GE Healthcare Ltd). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. The automation of synthesis of PET tracers performed on a synthesizer platform is limited by the number of available reagent slots. The method of the present invention permits a reduction in the number of chemicals required by removing the neutralizing agent. In another aspect, the present invention provides a cassette for carrying out the method of the invention, said cassette comprising:

(i) a vessel containing said protected precursor compound as defined herein;

(ii) means for eluting the vessel containing said protected precursor compound with a suitable source of F as defined herein;

(iii) means for deprotecting the ¹⁸F-labelled compound obtained following elution of the vessel containing said protected precursor compound with a suitable source of ¹⁸F; and,

(iv) an SPE column as defined herein suitable for trapping the deprotected ¹⁸F-labelled compound; with the proviso that a vessel containing a neutralization agent suitable for neutralizing the pH of said deprotected ¹⁸F-labelled compound is neither comprised in or in fluid connection with said cassette.

In the context of the cassette of the invention, a “neutralizing agent” is an acidic or an alkaline solution designed to neutralize the pH of, respectively an alkaline or an acidic solution comprising deprotected labelled ¹⁸F-labelled compound.

All the suitable, preferred, most preferred, especially preferred and most especially preferred embodiments of the precursor compound of Formula I, ¹⁸F-fluoride and the SPE cartridges that are presented herein in respect of the method of the invention also apply to the cassette of the invention.

The cassette of the invention may furthermore comprise:

(iv) an ion-exchange cartridge for removal of excess [¹⁸F]-fluoride.

### Diagnosis and Treatment Monitoring

¹⁸F-Fluorine (FLT) has been widely used as a oncology imaging probe for diagnostic positron emission tomography (PET). FLT is a nucleoside analog that enters cells and is phosphorylated by human thymidine kinase 1, but the 3′ substitution prevents further incorporation into DNA. However, its detailed accumulation mechanism remains unknown. Therefore, we investigated the chemical forms of FLT and their distributions in tumors using imaging mass spectrometry (IMS), which visualizes spatial distribution of chemical compositions based on molecular masses in tissue sections. Our radiochemical analysis revealed that most of the radioactivity in tumors existed as low-molecular-weight compounds with unknown chemical formulas, unlike observations made with conventional views, suggesting that the radioactivity distribution primarily reflected that of these unknown substances. The IMS analysis indicated that FLT and its reductive metabolites were nonspecifically distributed in the tumors in patterns not corresponding to the radioactivity distribution.

1-20. (canceled)

21. A method for determining the presence of, or susceptibility to, Acute Lymphoblastic Leukemia (ALL) in a mammalian subject with an in vivo imaging agent including an in vivo imaging moiety comprising a radiolabeled 3′ substituted nucleoside analog, the method comprising the steps of:
(i) administering to said mammalian subject a detectable quantity of said in vivo imaging agent that incliudes the in vivo imaging moiety comprising the 3′ substituted nucleoside analog;
(ii) allowing said administered in vivo imaging agent of step (i) to enter cells and become phosphorylated by human thymidine kinase 1, wherein the 3′ substitution of said nucleoside analog prevents further incorporation into DNA of said subject;
(iii) detecting signals emitted by said in vivo imaging agent of step (ii) that has entered said cells and has become phosphorylated using an in vivo imaging method;
(iv) generating an image representative of the location and/or amount of said signals; and,
(v) using the image generated in step (iv) to determine of the presence of, or susceptibility to, ALL.

22. The method of claim 21, wherein said in vivo imaging agent binds to a receptor site of a said cell entered in step (ii).

23. The method of claim 22, including producing a detectable species at said receptor site of said cell to which said in vivo imaging agent binds, when said in vivo imaging agent binds to said receptor site.

24. The method of claim 22, wherein producing a detectable species at said receptor site, corresponds with the presence of, or susceptibility to, Acute Lymphoblastic Leukemia (ALL) in said mammalian subject.

25. The method of claim 21, wherein detecting signals in step (iii) comprises scanning of said mammalian subject or portion thereof to detect activity or a change in activity in said cells wherein said in vivo imaging agent has become phosphorylated.

26. The method of claim 25, wherein said detecting signals is carried out using a suitable scanning method that detects the radiolabeled in vivo imaging agent.

27. The method of claim 25, wherein said detecting signals is carried out using magnetic resonance imaging (MRI), single photon emission tomography (SPECT), positron emission tomography (PET).

28. The method of claim 21, wherein said nucleoside analog is an ¹⁸F nucleoside analog.

29. The method of claim 21, wherein said nucleoside analog is 3′-deoxy-3′-¹⁸F-fluorothymidine (18F-FLT).

30. The method of claim 21, further comprising producing the in vivo imaging agent comprising said radiolabeled 3′ substituted nucleoside analog, wherein producing comprises the steps of:
(i) labelling a protected precursor compound with 18F;
(ii) deprotecting the 18F-labelled compound obtained in step (i) by hydrolysis;
(iii) diluting the deprotected 18F-labelled compound obtained in step (ii) with water;
(iv) trapping the deprotected 18F-labelled compound on a solid-phase extraction (SPE) column by passing the diluted solution obtained in step (iii) through said column;
(v) eluting the deprotected 18F-labelled compound from the SPE column; with the proviso that no neutralising step is carried out following the deprotection step;
wherein producing said in vivo imaging agent comprises producing at least a detectable quantity of said in vivo imaging agent that when administered to said mammalian subject enters the cells and becomes phosphorylated to produce a detectable species; and
wherein producing said in vivo imaging agent is carried out in temporal proximity to administering said in vivo imaging agent to said mammalian subject.

31. The method as defined in claim 30, wherein said deprotecting step (ii) is carried out by acid hydrolysis.

32. The method of claim 30, wherein said 18F-labelled compound is 3′-deoxy-3′-⁸F-fluorothymidine (18F-FLT).

33. The method of claim 32, wherein said protected precursor compound is a compound capable of receiving an 18F atom for labelling of the protective precursor compound.

34. The method of claim 33, including (a) adding a first volume of water to said deprotected ¹⁸F-labelled compound in step (iii) to obtain a first diluted solution.

35. The method of claim 34, including adding subsequent volumes of water to aliquots of said first diluted solution to obtain subsequent diluted solutions.

36. The method of claim 30, wherein said diluting step comprises:
(a) adding first volume of water to said deprotected 18F-labelled compound to obtain a first diluted solution, and, (b) adding subsequent volumes of water to aliquots of said first diluted solution to obtain subsequent diluted solutions.

37. The method of claim 30, wherein said SPE column is selected from Oasis HLB, tC18, and Strata X.

38. The method of claim 30, wherein said protected precursor compound is selected from the group consisting of:
(i) 1-(2′-Deoxy-2′-fluoro-β-L-arabinofuranosyl)-5-methyluracil (FMAU);
(ii) 3′-deoxy-3′-fluorothymidine (FLT);
(iii) 1-(2′-Deoxy-2′-[18F]fluoro-1-β-D-Arabinofuranosyl)-5-Bromouracil (FBAU);
(iv) thymidine.

39. The method of claim 30, which is automated.
